(19) United States
(12) Patent Application Publication
(10) Pub. No.: US 2013/0012745 A1
(43) Pub. Date: Jan. 10, 2013

(54) METHOD AND ARRANGEMENT FOR THE PRODUCTION OF HYDROCARBON COMPONENTS

(75) Inventors: Pekka KNUUTTILA, Porvoo (FI); Jaakko NOUSIAINEN, Lappeenranta (FI)

(73) Assignee: UPM-Kymmene Corporation, Helsinki (FI)

(21) Appl. No.: 13/542,322

(22) Filed: Jul. 5, 2012

Related U.S. Application Data
(60) Provisional application No. 61/504,841, filed on Jul. 6, 2011.

(30) Foreign Application Priority Data
Jul. 7, 2011 (FI) 20115725

(51) Int. Cl. C10G 3/00; B01J 8/04; B01J 19/24; C07C 1/22

(52) U.S. Cl. 585/240; 585/332; 422/630; 422/649; 422/632

(57) ABSTRACT

The invention relates to method for producing hydrocarbon components comprising isoparaffins from feedstock of biological origin comprising linear unsaturated fatty acids to produce diesel fuel components comprising the steps of a) converting at least part of linear unsaturated fatty acids comprised in the feedstock to corresponding branched fatty acids, and b) hydrodeoxygenating the said branched fatty acids and remaining linear fatty acids to corresponding isoparaffins and n-paraffins. The invention further relates to an arrangement for implementing the method of the invention.

### FIELD OF THE INVENTION

The present invention is in the field of renewable fuels and biofuels and relates to the production of hydrocarbons from biological raw materials for the production of biodiesel and components thereof. More particularly the invention relates to a method and apparatus for producing isoparaffins from feedstock of biological origin.

### BACKGROUND OF THE INVENTION

Production of hydrocarbon components for biofuel components and biofuels from biological raw materials is of increasing interest due to hope to replace non-renewable fossil raw materials with renewable starting materials. Several methods for producing fuels from biological raw materials are known in the art.

Conventional methods for producing biodiesel involve hydrotreating triglycerides and free fatty acids to n-paraffins. However, biodiesel produced in this manner has poor cold flow properties, such as cloud point and pour point, as compared to petroleum diesel although the cetane value is higher. In an attempt to overcome these problems, several methods have been reported for the production of biofuel components having high cetane number and good cold flow properties.

Middle distillates have been produced from vegetable oils by hydrogenating fatty acids and triglycerides of the vegetable oil to n-paraffins followed by isomerization to obtain branched paraffins. The presence of branched chain paraffins in biofuels is known to enhance its cold flow and physical properties.

One of the problems associated with these methods is the use of noble metal catalysts in the isomeration step. They are very expensive and highly sensitive to catalyst poisons. This is of high relevance when biological raw materials are exploited as they often contain heteroatoms, oxygen, sulphur, nitrogen, and/or phosphorus, which are catalysts poisons and inhibitors of noble metal catalysts.

Branched hydrocarbons for the production of lubricants have been produced by a process, which comprises the steps of isomeration, ketonisation and hydrodeoxygenation.

### BRIEF DESCRIPTION OF THE INVENTION

An object of the present invention is to provide a novel solution for producing biofuel components comprising branched hydrocarbons by treating feedstock of biological origin for producing biodiesel and biodiesel components of excellent quality. The objects of the invention are achieved by a method and an arrangement, which are characterized by what is stated in the independent claims. The preferred embodiments of the invention are disclosed in the dependent claims.

An advantage of the present invention is that it provides an efficient and cost effective solution for producing biodiesel and/or biodiesel components. Furthermore, the method according to the present invention yields products of excellent quality in terms of low temperature properties. Since a hydroisomerization step following a hydrodeoxygenation (HDO) step of a conventional process can be avoided by the method of the invention, utilization of costly isomeration catalyst(s) comprising noble metals is not required. Yet a further advantage of the method of the invention is that it allows the use of feedstock containing impurities that are harmful to conventional catalytic processes.

### BRIEF DESCRIPTION OF THE DRAWINGS

In the following the invention will be described in greater detail by means of preferred embodiments with reference to the attached drawings, in which

FIG. 1 shows schematically a flow chart of the method of the present invention for producing hydrocarbons comprising isoparaffins from feedstock of biological origin.

FIG. 2 shows schematically a first embodiment of the arrangement of the invention for producing hydrocarbons comprising isoparaffins from feedstock of biological origin;

FIG. 3 shows schematically a second embodiment of the arrangement of the invention for producing hydrocarbons comprising isoparaffins from feedstock of biological origin.

### DETAILED DESCRIPTION OF THE INVENTION

It was surprisingly found that high quality biodiesel and biodiesel components may be produced by the method of the invention, wherein unsaturated fatty acids comprised in a feedstock of biological origin are transformed to branched saturated paraffins, i.e. isoparaffins, prior to a hydrotreatment step.

In this specification and claims the term “biofuel” refers to renewable fuel, i.e. fuel made directly, via catalytic hydrotreatment, from renewable feedstock. The feedstock may be purified before it is used in the process, but it is not deliberately treated to its derivatives.

In this specification and claims the term “biodiesel” refers to renewable diesel fuel, i.e. diesel made directly, via catalytic hydrotreatment, from renewable feedstock. The feedstock may be purified before it is used in the process, but it is not deliberately treated to its derivatives. The meaning of the term “biodiesel” must not be mixed with fatty acid ethyl esters of vegetable oils which are commonly classified as biodiesels.

The present invention relates to a method for producing hydrocarbon components comprising isoparaffins from feedstock of biological origin, comprising the steps of

a) converting at least part of linear unsaturated fatty acids comprised in the feedstock to corresponding branched fatty acids,

b) hydrodeoxygenating said branched fatty acids and remaining linear unsaturated fatty acid to corresponding isoparaffins and n-paraffins.

In accordance with the present invention step a) is performed before step b).

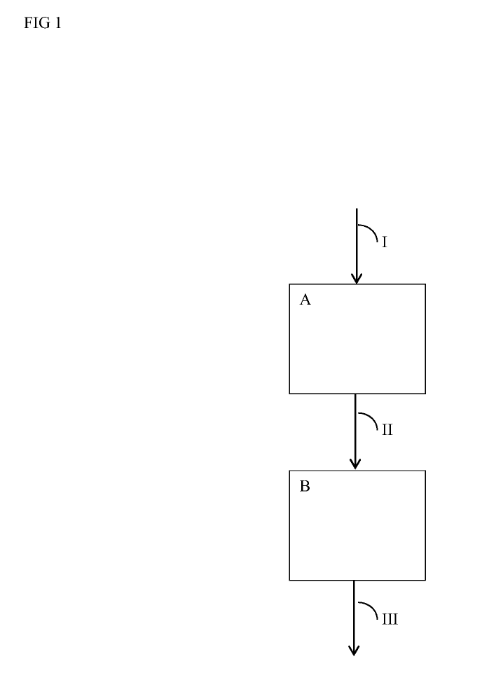

Referring to FIG. 1, in an embodiment of the method of the invention feedstock of biological origin I comprising unsaturated fatty acids is provided to a converting step A. In converting step A the feedstock I is reacted in the presence of a conversion catalyst and under suitable reaction conditions to form a first effluent II comprising branched fatty acids. The said first effluent II is then supplied to a hydrodeoxygenating step B and where the effluent is reacted in the presence of a hydrotreating catalyst and under suitable reaction conditions to form a second effluent III comprising isoparaffins.

The present invention further relates to an arrangement for producing hydrocarbon components comprising isoparaffins from feedstock of biological origin, wherein the arrangement is arranged to transform at least part of unsaturated fatty acids comprised in the feedstock to corresponding branched fatty acids, and then to hydrodeoxygenate said branched fatty acids and remaining unsaturated fatty acids to corresponding branched and linear paraffins for producing biodiesel fuel components.

In accordance with one embodiment of the arrangement of the present invention, the said arrangement comprises

one or more converting units for receiving feedstock of biological origin and subjecting said feedstock to conversion reactions to produce first effluent comprising branched fatty acids,

one or more hydrotreating units arranged after the converting unit(s) for receiving said first effluent and subjecting said first effluent to hydrotreatment in the presence of hydrogen containing gas to produce a second effluent comprising isoparaffins, and

one or more sources of hydrogen (or hydrogen containing) gas connected to the hydrotreating unit for providing said gas,

wherein the feedstock is arranged to be supplied to the converting unit, the first effluent is arranged to be supplied from the converting unit to the hydrotreating unit, and the second effluent is arranged to be recovered from the hydrotreating unit.

The converting unit and the hydrotreating unit according to the present invention may each be independently implemented by a catalyst bed comprising one or more catalyst layers, preferably 1 to 3 catalyst layers. In case two or more catalyst layers are used in a catalyst bed and two or more conversion catalysts/hydrotreating catalysts are used, the ratio(s) of the two (or more) catalysts in the individual catalyst layers may be same or different. The converting unit(s) and the hydrotreating unit(s) may be arranged in a same pressure vessel or in separate pressure vessels.

Hydrogen (or hydrogen containing) gas can be supplied to the arrangement suitably downstream or upstream to feedstock and/or effluent stream.

The converting unit(s) and the hydrotreating unit(s) may together be implemented by a fixed bed reactor, preferably a trickle-bed reactor (TBR), comprising two or more catalyst beds.

One or more inert layer(s) may optionally be arranged between a converting unit and a hydrotreating unit, and/or any two catalyst beds. Preferably an inert layer is arranged between a converting unit and a hydrotreating unit. The inert layer may be introduced to separate the catalyst layers from each other and/or allow heating or cooling of the effluent between the catalyst beds. Optionally an inert layer may be arranged before the first converting unit. Preferably such inert layer is a distribution layer. The purpose of said distribution layer is to establish an even liquid distribution across the catalyst beds. A preheating unit may optionally be arranged before the converting unit and/or between a converting unit and a hydrotreating unit.

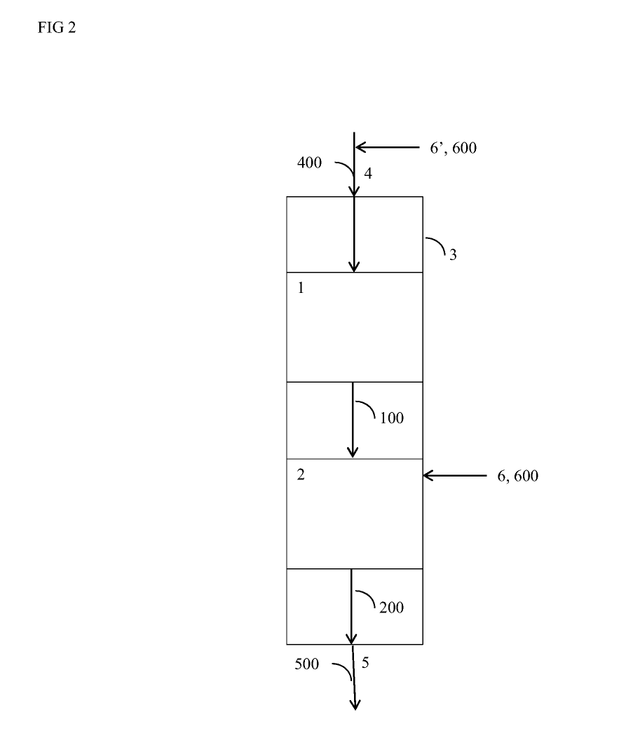

FIG. 2 shows schematically a preferred arrangement in accordance with the present invention. The arrangement comprises a conversion catalyst bed 1, and a hydrotreating catalyst bed 2 arranged successively in a reactor vessel 3 having an inlet 4 for supplying feedstock 400, an outlet 5 for recovering product stream 500, and a line 6 for supplying hydrogen or hydrogen containing gas 600.

With reference to FIG. 2, feedstock of biological origin 400 is supplied to the reaction vessel 3 through inlet 4. Hydrogen (or hydrogen containing) gas 600 may enter reactor 3 through line 6 or line 6′. The feedstock 40 then contacts the conversion catalyst bed 1 where it is subjected to a conversion reaction converting at least part of the comprised unsaturated fatty acids to corresponding branched fatty acids in order to provide an effluent 100 comprising said branched fatty acids. The said effluent 100 then flows downwards and contacts the hydrotreating catalyst bed 2 where it is subjected to hydrotreating reactions in the presence of 600 converting at least part of the said branched fatty acids to isoparaffins in order to provide product effluent 200 comprising desired product. The desired product stream is recovered from reactor 3 via the outlet 5 as product 500.

It is to be understood that line 6 supplying the hydrogen (or hydrogen containing) gas 600 may be divided, if desired, in any suitable way to supply the said gas 600 to where it is required, for example, so that one part of is connected to the top of the hydrotreating catalyst bed 2 and second part is connected to the middle of the catalyst bed 2, or in between any two catalyst layers of the said catalyst bed 2. A separating unit, such as a distillation unit, may be incorporated into the outlet 5, in order to remove undesirable components from product effluent 200 in order to provide the product stream 500 recovered from reactor 3. Furthermore, a purifying unit, such as a filtering unit, may be incorporated into the inlet 4 in order to remove undesirable components from feedstock 400 supplied to reactor 3.

The reactor may be operated under any suitable conditions depending upon the nature of the feed and the desired product. The temperature of the conversion catalyst bed and the hydrotreating catalyst bed may be same or different. One or more suitable conversion catalysts may be incorporated into the conversion catalyst bed 1 and one or more suitable hydrotreating catalysts may be incorporated into the hydrotreating catalyst bed 2.

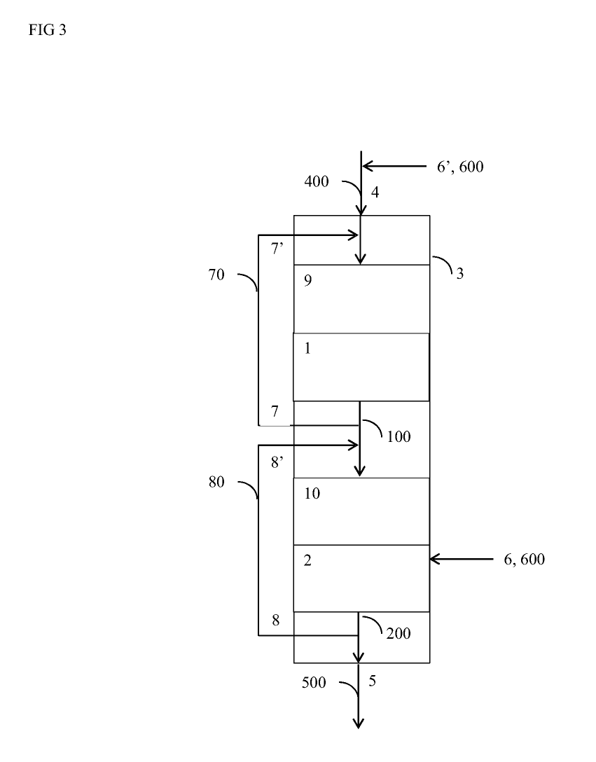

An alternative embodiment of the arrangement according to the present invention is illustrated schematically in FIG. 3. In FIG. 3, like components are designated by the same reference numerals as used in FIG. 2. The arrangement further comprises an outlet 7 and an inlet 7′ arranged to reactor 3 and connected via a recycle line 70, and an outlet 8 and an inlet 8′ arranged to reactor 3 and connected via a recycle line 80.

Part or all of the effluent 100 which comprises unreacted unsaturated fatty acids may be withdrawn from reactor 3 through outlet 7 prior to entry into the hydrotreating catalyst bed and recycled via line 70 through inlet 7′ to the reactor 3 for re-contacting the conversion catalyst bed 1. Although FIG. 3 shows inlet 7′ connected to inlet 4, the recycled material can alternatively be supplied separately to reactor 3. Part or all of the effluent 200 which comprises unreacted branched fatty acids may be withdrawn from reactor 3 though outlet 8 and recycled via line 80 though inlet 8′ to the reactor 3 after the conversion catalyst bed 1 for re-contacting the hydrotreating catalyst bed 2. Although FIG. 3 shows inlet 8′ connecting to reactor 3 in between the conversion catalyst bed 1 and the hydrotreating catalyst bed 2, and thus the recycled effluent 200 joining effluent 100, it is to be understood that the recycled material 200 can alternatively be supplied separately to the hydrotreating catalyst bed 2.

FIG. 3 additionally shows an inert top layer 9 arranged directly before the conversion catalyst bed 1 and an inert layer 10 interposed between the conversion catalyst bed 1 and the hydrotreating catalyst bed 2. A first distribution layer and/or a first preheating unit may be incorporated into the inert top layer 9. The distribution layer functions to evenly distribute the feedstock 400 to conversion catalyst bed 1 and the preheating unit functions to adjust the temperature of the feedstock 100 to a desired level prior to entry into the conversion catalyst bed 1. Similarly, a second distribution layer and a second preheating unit may be incorporated in the inert layer 10. FIG. 3 further shows an inlet 11 connected to reactor 3 in between the conversion catalyst bed 1 and the hydrotreating catalyst bed 2 for supplying hydrogen sulphide or hydrogen sulphide releasing source 110 and mixing it with effluent 100. It is to be understood that the outlet 11 may alternatively be arranged to directly connect the hydrotreating catalyst bed 2 at one or more suitable locations.

The gaps shown in FIG. 2 and FIG. 3 between the catalyst bed do not necessary implicate empty spaces between the applicable catalyst layers and/or inert layers, but are present merely for the sake of the clarity of the drawings.

The present invention further relates to use of one or more conversion catalyst and one or more hydrotreating catalyst in converting at least part of unsaturated fatty acids comprised in a feedstock of biological origin to corresponding branched fatty acids and the hydrodeoxygenating said branched fatty acids to corresponding isoparaffins.

### Feedstock

The feedstock of the present invention comprises unsaturated and/or polyunsaturated free fatty acids having between 4 and 28 carbon atoms and one or more double bond in the carbon chain, and/or triglycerides, esters or salts thereof, preferably free fatty acids. The fatty acid (FA) composition of several oils and fats is presented in Table 1.

TABLE 1

| | unsaturated FA | oleic acid | linoleic acid |
|---|---|---|---|
| Fat or oil | (w/w) | (w/w) | (w/w) |
| almond oil | 93% | 62% | 24% |
| corn oil | 85% | 29% | 52% |
| lard | 56-62% | 44-47% | 6-10% |
| olive oil | 72-90% | 55-83% | 3.5-21% |
| palm oil | 49% | 39% | 10% |
| canola oil | 95% | 61% | 21% |
| sunflower oil | 83% | — | 68% |
| wheat germ oil | 77% | 14% | 55% |
| soya oil | 81% | 23% | 51% |

If not otherwise indicated, the term “fatty acid” as used herein and hereafter refers to carboxylic acids having a long, unbranched, aliphatic hydrocarbon chain which is either saturated or unsaturated. Most naturally occurring fatty acids have a chain of an even number of carbon atoms, suitably from 4 to 28. Unsaturated fatty acids have one or more double bonds between carbon atoms of the hydrocarbon backbone. Fatty acids bearing more than one double bond are often referred to as polyunsaturated fatty acids. However the term “unsaturated fatty acids” as used herein is understood to refer to both monounsaturated and polyunsaturated fatty acids. The two carbon atoms bound next to either side of the double bond can occur in a cis or trans configuration. The differences in configuration may play role in the reactions of the various types of unsaturated fatty acids. The fatty acids of the present invention may be in the form of free fatty acids, or triglycerides, esters, or salts thereof, preferably free fatty acids.

Fatty acids are widely distributed in plant oils as well as other oils and fats of biological origin. Two examples of unsaturated fatty acids are oleic acid (OA) and linoleic acid (LA). The term “linoleic acid” as used herein and hereafter is understood to refer to a carboxylic acid with an 18-carbon chain and two cis double bonds. The term “oleic acid” as used herein and hereafter is understood to refer to monounsaturated omega-9 fatty acid found in various animal and vegetable fats. The structures of OA and LA are shown below.

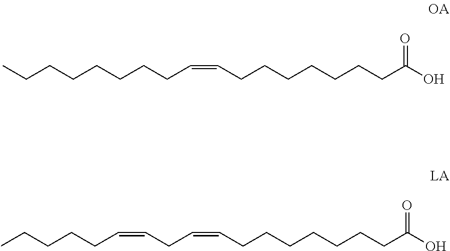 [SMILES: CCCCCCCC/C=C\CCCCCCCC(=O)O ; CCCCC/C=C\C/C=C\CCCCCCCC(=O)O]

The feedstock of the present invention preferably comprises at least 5% by weight (w/w) of unsaturated and/or polyunsaturated fatty acids, more preferably over 50% (w/w), most preferably from 50 to 90% (w/w). The carbon number of the said fatty acid is advantageously 12 to 28, preferably 16 to 24. In an embodiment of the invention the feedstock comprises linoleic acid and/or oleic acid. Oils and fats comprising linoleic and/or oleic acids are particularly suitable for producing diesel oil components as the chain length for diesel components is suitably in the range of C₉-C₂₀.

The feedstock of the present invention may consist of plant, animal, and fish oils and fats, and mixtures thereof. Examples of suitable oils and fats containing saturated and/or polyunsaturated fatty acids and/or their derivatives include, but are not limited to, almond oil, babassu oil, ben oil, butter, canola oil, castor oil, chicken fat, colza oil, corn oil, cottonseed oil, crude tall oil, false flax oil, fish oils, grape seed oil, hemp oil, honge oil, jatropha seed oil, jojoba oil, lard, linseed oil, mustard oil, olive oil, palm oil, palm kernel oil, peanut oil, pecan oil, pistachio oil, poppyseed oil, pumpkin seed oil, rapeseed oil, rendered animal fats, rice bran oil, sea buckthorn oil, sesame oil, sunflower oil, soybean oil, tall oil, tallow, tung oil, turkey fat, walnut oil, and wheat germ oil.

An example of a readily available non-edible plant oil comprising linoleic acid is tall oil. Crude tall oil (CTO) is a by-product of the Kraft process of wood pulp manufacture when pulping mainly coniferous trees. Normally CTO contains unsaponifiables (5 to 30%), resin acids (20 to 50%), fatty acids (mainly palmitic acid, oleic acid and linoleic acid) (20 to 70%), fatty alcohols, some sterols, and other alkyl hydrocarbon derivatives. Tall oil fatty acid (TOFA) consisting mostly of oleic acid can be obtained from crude tall oil by fractional distillation. In a preferred embodiment of the present invention the feedstock comprises crude tall oil, tall oil, and/or TOFA. In an advantageous embodiment of the present invention the feedstock is crude tall oil.

In accordance with the present invention the feedstock may contain sulphur, nitrogen, aromatic impurities, and other impurities.

The feedstock may be subjected, prior to step a) of the present invention to one or more pre-processing steps, for example to a purification step to remove contaminants such as metals from the feedstock. The preprocessing step(s) may be accomplished with suitable known methods. These methods include, but are not limited to, thermal and/or chemical treatments known to the person skilled in the art, such as washing, filtering, distillation, degumming and depitching.

If necessary, free fatty acids may be produced from corresponding triglycerides, esters, and/or metals comprised in the feedstock prior to the conversion step by standard processes known to a person of ordinary skill in the art. Examples of these processes include, but are not limited to, vapour splitting of triglycerides, basic hydrolysis, and enzymatic hydrolysis.

### Conversion Step

In the conversion step, step a), of the method according to the present invention the feedstock, which may be pre-processed, is subjected to conditions suitable for converting at least part of linear unsaturated fatty acids comprised in the feedstock to corresponding branched fatty acids. In accordance with the present invention an effluent comprising branched fatty acids is produced from the feedstock of biological origin in step a) the said branched fatty acids may be saturated or unsaturated depending on the nature of the parent fatty acid and/or the reaction conditions. Preferably at least 30% of the said fatty acids are converted to corresponding branched fatty acids, more preferably 30 to 50%, most preferably 50 to 80%. The said effluent may additionally comprise unreacted linear unsaturated fatty acids and/or other unreacted compounds present in the feedstock.

In the conversion step of the method the unsaturated fatty acids undergo a chemical transformation, referred herein as isomerization, rendering branched fatty acids with corresponding carbon number as products. Whilst we do not intend to be bound by any particular theory, the isomerization likely proceeds via a Brønsted acid catalysed carbocation formation (i) across a double bond of the unsaturated fatty acid and subsequent rearrangement (ii) of the hydrocarbon skeleton of the said fatty acid rendering isomerized (branched) fatty acids, for example, as follows

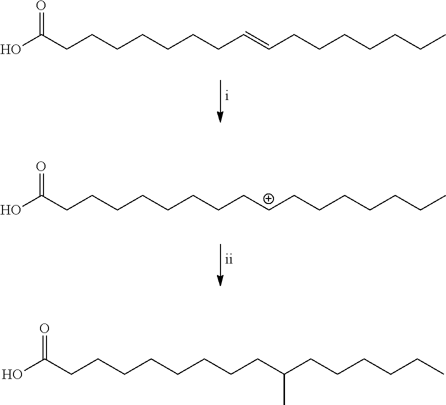

The carbocation formation may also lead to catalytic cracking to give shorter alkanes, alkenes and/or fatty acids. However, it is possible to favour the isomerization mechanism over the cracking mechanism by careful selection of the acid catalyst and reaction conditions.

The Brønsted acid catalysis may also lead to double bond migration (DBM) and/or cis-trans isomerisation of the double bond of the unsaturated fatty acid prior to the carbocation formation. This will lead to the formation of mixtures of regioisomers of corresponding branched fatty acids. Branches in the middle of the hydrocarbon skeleton will lower the cloud point more than those close to the ends of the chain.

Polyunsaturated fatty acids may undergo the carbocation mediated isomerization reaction at one or more double bond sites rendering products with multiple numbers of branches. The number of branches is limited by the number of double bonds in the fatty acids comprised in the feedstock. The cloud point will be lowered by the increasing number of branches, however also the cetane index is reduced.

The conversion step may be accomplished utilizing a conversion catalyst having a Brønsted acid functionality. The conversion catalyst of the present invention may be amorphous silica alumina or a zeolite, silica aluminium phosphate (SAPO), and/or aluminium phosphate (AIPO). Advantageously the said catalyst is a mono-dimensional zeolite molecular sieve.

The term “zeolite” as used herein and hereafter refers to crystalline microporous, aluminosilicate minerals having a precisely defined pore system. The pores can be adjusted to precisely determined uniform openings allowing for molecules smaller than its pore diameter to be adsorbed whilst excluding lager molecules. The distribution profile of the location of the branches in the hydrocarbon skeleton of the fatty acid may be influenced by the choice of the zeolite catalyst. The long hydrocarbon chain of the unsaturated fatty acid bearing the double bond will diffuse into the zeolite in order to interact with the Brønsted acid functionality buried inside the zeolite framework while the carboxylic acid functionality remains on the outside.

The physical and chemical interactions between the reacting molecule and the zeolite determine the capability of the hydrocarbon chain to enter into the zeolite framework. Thus the optimal loading, channel size and shape (i.e. pore structure), and location of the Brønsted acid functionality of the zeolite depend on the nature of the diffusing molecule i.e. the unsaturated fatty acids comprised in the feedstock and the desired outcome of the product and vice versa. The Brønsted acid strength of a zeolite is determined by the aluminium level of the framework. Examples of suitable zeolites include, but are not limited to, pentasils and modernites. Preferably, the conversion catalyst is selected from ZSM-5, ZSM-22, ZSM-23, SAPO-11, SAPO-41 and mixtures thereof.

In accordance with step a) of the method according to the present invention the feedstock is contacted with one or more Brønsted acid catalysts. The temperature range may be, for example, from 100 to 500° C., preferably from 200 to 400° C. and most preferably 250 to 290° C. The temperature of the conversion step depends on the desired specification of the product and/or the nature of the catalyst and/or the nature of the feedstock. The operating pressure may vary between 0 and 150 bars, more preferably between 10 and 90 bars. In a case a fixed bed reactor is used the hourly weight hourly space velocity (WHSV), defined as the weight of feed per hour per unit weight of catalyst loaded in the reactor, is 0.1 to 100 h⁻¹, preferably 0.1 to 20 h⁻¹ most preferably 0.3 to 10 h⁻¹.

In accordance with the present invention the effluent from step a) is supplied to step b) of the present invention. Advantageously, step a) and step b) are performed within one pressure vessel. If desired, the said effluent from step a) may be subjected to one or more purification steps for removing any undesired impurities prior to entry to step b). Furthermore, the temperature of the said effluent may be adjusted to desired level before it is supplied to step b). Further in accordance of the present invention part or all of the said effluent may be recycled to step a) or to the feedstock for increasing the conversion and/or selectivity of step a) prior to entry to step b).

### Hydrotreating Step

In accordance with the hydrotreating step, step b), of the method of the invention the branched fatty acids obtained in the first step are hydrodeoxygenated (HDO) to the corresponding branched paraffins. Furthermore, in accordance with the step b) of the method of the invention, the linear saturated and unsaturated fatty acids comprised in the effluent from step a) may advantageously be hydrodeoxygenated and, when required, hydrogenated to the corresponding saturated n-paraffins. Further in accordance with the present invention, in the step b) the effluent from step a) is subjected to hydrotreatment to obtain product effluent comprising isoparaffins.

The terms “isoparaffin”, “branched paraffin”, and “branched saturated hydrocarbons” as used herein and hereafter refer to branched alkanes, all meaning the same.

The term “hydrotreatment” as used herein and hereafter refers to catalytic process suitably involving hydrodeoxygenation (HDO), hydrodesulphurisation (HDS), hydrodenitrogenation (HDN), hydrodehalogenation (HDH) reactions. Hydrotreatment may also result to decarboxylation and/or decarbonylation of carbonyl containing organic compounds as well as hydrogenation of carbon-carbon double bonds of unsaturated organic compounds, ring opening of cyclic and polycyclic organic compounds, and, in some circumstances, hydrocracking of organic compounds.

HDO is understood to remove oxygen from oxygen containing organic molecules, such as fatty acids, aldehydes, ketones, alcohols, and esters, as water. HDS is understood to remove sulphur from sulphur containing organic molecules as dihydrogen sulphide (H₂S). HDN is understood to remove nitrogen from nitrogen containing compounds as ammonia (NH₃). HDH is understood to remove halogens from halogen containing compounds as corresponding hydrohalic acids.

In accordance with the present invention the hydrotreating step may be accomplished utilizing a hydrotreating catalyst. In step b) of the method according the present invention the effluent from step a) is contacted with a hydrotreating catalyst. Examples of suitable hydrotreating catalysts include, but are not limited to, catalyst containing Group 6, Group 8, Group 9, and/or Group 10 metals of the IUPAC Periodic Table. Preferably, the hydrotreating catalyst is a supported monometallic or multiple metal combination catalyst of Ni, Co, W, Mo, or any combinations thereof, or a catalyst mixture thereof, wherein the support preferably is activated carbon, alumina, silica alumina, or silica. The metal portion of the catalyst may thus be NiMo, CoMo, NiW, NiCoMo, or any mixture thereof. In an embodiment of the present invention the hydrotreating catalyst is a NiMo catalyst supported on alumina. In a preferred embodiment of the present invention the hydrotreating catalyst is a NiW catalyst supported on alumina, silica alumina, or silica. In an advantageous embodiment of the present invention the hydrotreating catalyst further contains zeolite.

The operating conditions of the hydrotreating step depended on the desired specification of the product, the nature of the effluent from step a) and the nature of the hydrotreating catalyst. The temperature range may be, for example, from 100 to 500° C., preferably from 250 to 450° C., more preferably 300 to 410° C. The operating pressure may vary between 10 and 150 bars, more preferably between 20 and 100 bars. Advantageously the pressure of step b) is same as the pressure of step a). In a case a fixed bed reactor is used the hourly weight hourly space velocity (WHSV), defined as the weight of feed per hour per unit weight of catalyst loaded in the reactor, is 0.2 to 10 h⁻¹, preferably 0.5 to 3 h⁻¹. In accordance of the present invention the operating conditions of step a) and step b) may be same or different.

The amount of hydrogen gas needed in the hydrotreating step is determined by the amount and the nature of the effluent from step a). A suitable amount of hydrogen can be determined by a person having ordinary skills in the art.

It may be necessary to supply supplementary sulphur to the hydrotreating step to maintain the catalytic activity of the hydrotreating catalyst, depending on nature of the feedstock. If desired, supplementary sulphur may be supplied to the hydrotreating step. Said sulphur can be hydrogen sulphide (H₂S) and/or any sulphur containing compound that produces hydrogen sulphide in the process. Examples of suitably sulphur containing compounds include, but are not limited to, organic sulphur compounds, such as dimethyl disulphide. Additionally or alternatively, natural sulphur containing compounds present in raw tall oil may be utilized as supplementary sulphur sources in accordance with the invention.

The product effluent obtained according to method of the invention comprises isoparaffins. The said effluent may further comprise n-paraffins and/or other hydrocarbons. If desired, part or all of the product effluent from step b) may be recycled to step b) to increase the conversion and/or selectivity of step b) and/or to control the operating conditions.

Following step b) the product effluent may be subjected to conventional separation and/or purification steps known to a person having ordinary skills in the art.

It will be obvious to a person skilled in the art that, as the technology advances, the inventive concept can be implemented in various ways. The invention and its embodiments are not limited to the examples described above but may vary within the scope of the claims.

1. A method for producing hydrocarbon components comprising isoparaffins from feedstock of biological origin comprising linear unsaturated fatty acids to produce diesel fuel components, wherein the method comprises the steps of
a) converting at least part of linear unsaturated fatty acids comprised in the feedstock to corresponding branched fatty acids, and;
b) hydrodeoxygenating said branched fatty acids and remaining linear unsaturated fatty acids to corresponding paraffins and n-paraffins.

2. The method as claimed in claim 1, wherein an effluent comprising branched fatty acids is produced is step a) and the said effluent is then subjected to hydrotreating in step b).

3. The method as claimed in claim 1, wherein the feedstock is selected from plant oils and fats, animal fats, fish oils, and mixtures thereof.

4. The method as claimed in claim 3, wherein the feedstock is crude tall oil.

5. The method as claimed in claim 4, wherein the feedstock is subjected, prior to step a) to a purification step to remove metals from the feedstock.

6. The method as claimed in claim 1, wherein step a) is performed in the presence of a conversion catalyst having a Brønsted acid functionality.

7. The method as claimed in claim 6, wherein the conversion catalyst is selected from amorphous silica alumina, zeolites, silica aluminium phosphates (SAPO), aluminium phosphates (AIPO), silica alumina, silica aluminium phosphates (SAPOs), aluminium phosphates (AIPO) and mixtures thereof.

8. The method as claimed in claim 7, wherein the conversion catalyst is selected from ZSM-5, ZSM-22, ZSM-23, SAPO-11, SAPO-41 and mixtures thereof.

9. The method as claimed in claim 1, wherein step b) is performed in the presence of a hydrotreating catalyst containing Group 6, Group 8, Group 9, and/or Group 10 metals of the periodic table.

10. The method as claimed in claim 9, characterized in that the hydrotreating catalyst is selected from supported monometallic or multiple metal combination catalysts of Ni, Mo, Co, W, and any combinations thereof, and catalyst mixtures thereof; and the support is selected from activated carbon, alumina, silica, silica-alumina, and mixtures thereof.

11. The method as claimed in claim 10, wherein the hydrotreating catalyst is a NiW catalyst supported on alumina, silica alumina or silica.

12. The method as claimed in claim 11, wherein the hydrotreating catalyst further contains zeolite.

13. The method as claimed in claim 1, where in step a) the temperature is from 100 to 500° C., the operating pressure is between 0 and 150 bars, and the hourly weight hourly space velocity is 0.1 to 100 h⁻¹.

14. The method as claimed in claim 1, where in step b) temperature is from 100 to 500° C., the operating pressure is between 10 and 150 bars, and the hourly weight hourly space velocity is 0.2 to 10 h⁻¹.

15. An arrangement for producing hydrocarbon components from feedstock of biological origin, characterized in that the arrangement is arranged to transform at least part of linear unsaturated fatty acids comprised in the feedstock to corresponding branched fatty acids and then to hydrodeoxygenate said branched fatty acids and remaining linear unsaturated fatty acids to corresponding isoparaffins and n-paraffins for producing biodiesel fuel components.

16. The arrangement as claimed in claim 15, characterized in comprising
one or more converting units for receiving feedstock of biological origin and subjecting said feedstock to conversion reactions to produce first effluent comprising branched fatty acids,
one or more hydrotreating units arranged after the converting unit(s) for receiving said first effluent and subjecting said first effluent to hydrotreatment in the presence of hydrogen containing gas to produce a second effluent comprising isoparaffins, and
one or more sources of hydrogen containing gas connected to the hydrotreating unit for providing said gas,
wherein the feedstock is arranged to be supplied to the converting unit, the first effluent is arranged to be supplied from the converting unit to the hydrotreating unit, and the second effluent is arranged to be recovered from the hydrotreating unit.

17. The arrangement as claimed in claim 16, characterized in that the converting unit(s) and the hydrotreating unit(s) are arranged in same pressure vessel.

18. The arrangement as claimed in claim 16, characterized in that the converting unit and the hydrotreating unit are each independently a catalyst bed comprising one more catalyst layers.

19. The arrangement as claimed in claim 16, characterized in that one or more inert layers are arranged before the converting unit and/or between the converting unit and the hydrotreating unit.

20. The arrangement as claimed in claim 16, characterized in that a preheating unit is be arranged before the converting unit and/or between the converting unit and the hydrotreating unit.

21. The arrangement as claimed in claim 16, characterized in that the converting unit and the hydrotreating unit are together a fixed bed reactor, preferably a trickle-bed reactor (TBR), comprising two or more catalyst beds.
